(19) United States
(12) Patent Application Publication
(10) Pub. No.: US 2020/0178827 A1
(43) Pub. Date: Jun. 11, 2020

(54) NOISE REMOVAL IN MAGNETOMETER FOR MEDICAL USE

(71) Applicant: CREAVO MEDICAL TECHNOLOGIES LIMITED, Leeds (GB)

(72) Inventors: Abbas Ahmad Al-Shimary, Leeds (GB); David Diamante Dimambro, Leeds (GB); Benjamin Thomas Hornsby Varcoe, Leeds (GB); Richard Theodore Grant, Leeds (GB)

(73) Assignee: CREAVO MEDICAL TECHNOLOGIES LIMITED, Leeds (GB)

(21) Appl. No.: 16/478,115

(22) PCT Filed: Aug. 3, 2018
(86) PCT No.: PCT/GB2018/052223

§ 371 Date: Jul. 15, 2019

(30) Foreign Application Priority Data
Aug. 18, 2017 (GB) 1713285.3

(51) Int. Cl. A61B 5/04; A61B 5/00

(52) U.S. Cl. CPC A61B 5/04007; A61B 5/7207; A61B 5/7225; A61B 5/7285; A61B 5/6874; A61B 5/6869; A61B 5/6868; A61B 5/725

(57) ABSTRACT

A method of using a magnetometer system to analyse the magnetic field of a region of a subject's body is disclosed. The method comprises using one or more detectors to detect the time varying magnetic field of a region of a subject's body, filtering a signal or signals from the one or more detectors using a filter or filters, and using the filtered signal or signals to analyse the magnetic field generated by the region of a subject's body. The filter or filters is configured to attenuate noise in the signal or signals that is synchronised with motion of the region of the subject's body such as ballistocardiographic noise.

The present invention relates to methods and apparatus for medical magnetometry, and in particular to methods and apparatus for processing a signal from a magnetometer for medical use, such as for use as a cardiac magnetometer.

It can be useful in many medical situations to be able to measure magnetic fields relating to or produced by the human body for diagnostic purposes. For example, the heart's magnetic field contains information that is not contained in an ECG (Electro-cardiogram), and so a magneto cardiogram scan can provide different and additional diagnostic information to a conventional ECG.

The Applicants have devised a portable magnetometer device that is intended for use, for example, in a medical environment such as a hospital or surgery without magnetic shielding, cryogenic cooling, etc. (as described in WO2014/006387).

The, e.g., medical environment can present a number of challenges for the acquisition of acceptable MCG data. In particular, noise from the medical environment can interfere with the desired signal. Such noise can often exceed the signal by orders of magnitudes, meaning that the removal of such noise is challenging.

The Applicants believe that there remains scope for improvements to the design and use of magnetometers for medical use, and in particular for cardio magnetic sensing and/or imaging.

According to a first aspect of the present invention, there is provided a method of using a magnetometer system to analyse the magnetic field of a region of a subject's body, the method comprising:

using one or more detectors to detect the time varying magnetic field of a region of a subject's body;

filtering a signal or signals from the one or more detectors using a filter or filters, wherein the filter or filters is configured to attenuate ballistocardiographic noise in the signal or signals; and

using the filtered signal or signals to analyse the magnetic field generated by the region of a subject's body.

According to a second aspect of the present invention, there is provided a magnetometer system for medical use, comprising:

one or more detectors for detecting the time varying magnetic field of a region of a subject's body; and

a filter or filters configured to filter a signal or signals from the one or more detectors, wherein the filter or filters is configured to attenuate ballistocardiographic noise in the signal or signals;

wherein the magnetometer system is configured to provide the filtered signal or signals for use to analyse the magnetic field generated by the region of the subject's body.

The present invention is concerned with a method of analysing the magnetic field of a region of a subject, such as their heart. In the present invention, one or more detectors are used to detect the time varying magnetic field of a region of a subject's body, and a signal or signals from the one or more detectors is or are filtered.

In contrast with existing arrangements, in the present invention, the filter or filters is configured to attenuate (e.g. separate or remove) ballistocardiographic (BCG) effects (noise) in the measured signal or signals. As will be described in more detail below, the Applicants have found that using a filter in the manner of the present invention is particularly beneficial for removing unwanted noise from the signal or signals.

In this regard, the Applicants have recognised, in particular, that in the case of medical magnetometry, e.g. performed in a medical environment such as a hospital, ambulance or surgery, where the subject is placed on a support structure such as a bed, motion of the subject's body, e.g. as the subject's heart beats, can cause the structure (e.g. bed) to move, i.e. vibrate. Since medical beds often have a steel frame, such motion can give rise to magnetic background noise which can be picked up by the magnetometer system. This “ballistocardiographic noise” or “bed noise” can corrupt the MCG signal to such a degree that it can be difficult or even impossible to extract useful medical information from the MCG signal.

The Applicants have furthermore recognised that such noise is not merely deterministic, and is instead in the form of “transient noise” (i.e. comprising an initial pulse due to coupling of the heartbeat to the support structure (e.g. bed) followed by decaying oscillations due to vibration of the system), and appears in a frequency range that overlaps with the frequency range of the signal from the heart. This transient noise is therefore challenging to remove.

On the other hand, it may not always be possible (or desirable) to avoid such noise, e.g. by using magnetic shielding and/or an electrically insulating and/or non-magnetic support structure such as a wooden bed. For example, electrically insulating (non-conductive) and/or non-magnetic beds may not be present in a medical environment such as a hospital, ambulance or surgery, or it may be undesirable to move a patient to such a bed, e.g. in a medical emergency or otherwise. As such, the Applicants have recognised that conventional approaches that attempt to avoid noise by using magnetic shielding and/or an electrically insulating (non-conductive) and/or non-magnetic support structure can be impractical in a medical environment.

The present invention accordingly lies firstly in the identification of this new type of “ballistocardiographic noise”, and secondly in the recognition that such noise can be successfully removed from the signal using the filter of the present invention. This accordingly facilitates the extraction of useful medical information from MCG signals in a “normal” medical environment, without resorting to the use of magnetic shielding and/or an electrically insulating (non-conductive) and/or non-magnetic support structure, i.e. even when a subject is supported by a structure comprising electrically conductive and/or magnetic material, e.g. when the subject is on a steel framed bed in a medical environment.

It will be appreciated therefore that the present invention provides an improved magnetometer system for medical use.

The magnetometer system of the present invention can be used in a normal hospital, ambulance, or surgery or other environment, without the need for magnetic shielding. Thus, in a particularly preferred embodiment, the methods of the present invention comprise using the magnetometer system to detect the magnetic field of a subject's heart (or other body region) in a non-magnetically shielded environment (and without the use of magnetic shielding). Correspondingly, the magnetometer system of the present invention preferably does not comprise (other than comprises) magnetic shielding.

It should be noted that, as used herein, a “magnetically shielded environment” is intended to include arrangements where a magnetometer is either arranged in a shielded room or enclosure. In such arrangements, both the subject being measured and the magnetometer are contained within the same shielded room or enclosure. By contrast, as used herein, a magnetometer may be considered to be in a “non-magnetically shielded environment” where no external piece or pieces of apparatus are used to protect the subject being measured, nor the magnetometer doing the measuring.

As described above, a particular advantage of the magnetometer system of the present invention is that it can be used to detect the magnetic field of a region of a subject's body when the subject is supported by (is on) a structure such as a chair or a (hospital) bed, that comprises electrically conductive, e.g. metallic, ferrous and/or magnetic material, such as a steel frame.

Accordingly, in a particularly preferred embodiment, the methods of the present invention comprise using the magnetometer system to detect the magnetic field of a subject's heart (or other body region) when the subject is supported by a structure comprising electrically conductive, e.g. metallic, ferrous and/or magnetic material, e.g. when the subject is on a (hospital) bed or chair that has a frame formed from electrically conductive, e.g. metallic, ferrous and/or magnetic material. Correspondingly, the magnetometer system of the present invention preferably comprises a support structure for supporting the subject's body that comprises electrically conductive, e.g. metallic, ferrous and/or magnetic material.

The magnetometer system of the present invention can be used as a system and probe to detect any desired magnetic field produced by a subject (by the human (or animal) body). It is preferably used to detect (and analyse) the time varying magnetic field of (or produced by) a region of the subject's body, such as their bladder, abdomen, chest or heart, head or brain, muscle(s), womb or one or more foetuses. Thus it may be, and is preferably, used to detect magnetic fields relating to the bladder, pregnancy, muscle activity, the brain, or the heart. In a preferred embodiment, the magnetometer is used for (and configured for) one or more of: magnetocardiography, magnetoencephalography, analysis and detection of bladder conditions (e.g. overactive bladder), analysis and detection of foetal abnormalities, and detection and analysis of pre-term labour.

In a particularly preferred embodiment the magnetometer is used as a cardiac magnetometer and to detect and analyse the magnetic field of a subject's heart.

Thus, according to another aspect of the present invention there is provided a method of analysing the magnetic field of a subject's heart, the method comprising:

using one or more detectors to detect the time varying magnetic field of a subject's heart;

filtering a signal or signals from the one or more detectors using a filter or filters, wherein the filter or filters is configured to attenuate ballistocardiographic noise in the signal or signals; and

using the filtered signal or signals to analyse the magnetic field generated by the subject's heart.

According to another aspect of the present invention, there is provided a cardiac magnetometer system for analysing the magnetic field of a subject's heart, comprising:

one or more detectors for detecting the time varying magnetic field of a subject's heart; and

a filter or filters configured to filter a signal or signals from the one or more detectors, wherein the filter or filters is configured to attenuate ballistocardiographic noise in the signal or signals;

wherein the magnetometer system is configured to provide the filtered signal or signals for use to analyse the magnetic field generated by the subject's heart.

As will be appreciated by those skilled in the art, these aspects of the present invention can and preferably do include any one or more or all of the preferred and optional features of the invention described herein, as appropriate.

The one or more detectors of the present invention may be configured to detect the time varying magnetic field of a region of a subject's body in any suitable and desired manner.

The magnetometer system of the present invention may comprise a single detector. In this case, the detector may be positioned appropriately over a subject (e.g. a subject's chest or other region of the subject's body) to take readings from a suitable (single) sampling position for the region of the subject's body in question. Alternatively, the detector may be moved over the subject (e.g. the subject's chest) to take readings from plural different sampling positions in use.

However, in one preferred embodiment, the magnetometer system comprises plural detectors, e.g. and preferably at least 7, e.g. 7-500 (or more), preferably at least 16, e.g. 16-500 (or more) detectors.

Where the magnetometer system comprises plural detectors, some or all of the detectors may be arranged in a two or three dimensional array, e.g. and preferably at least 7, preferably at least 16, detectors arranged in a two or three dimensional array. In this case, the or each detector array is preferably configured such that when positioned appropriately over a subject (e.g. a subject's chest or other region of the subject's body) the detector array can take readings from a suitable set of sampling positions without the need to further move the array over the subject.

The or each array can have any desired configuration, such as being a regular or irregular array, a hexagonal, rectangular or circular array (e.g. formed of concentric circles), etc.

The number and/or configuration of detectors in the or each array is preferably selected so as to provide an appropriate number of sampling points and/or an appropriate coverage for the region of the subject's body in question.

In a preferred embodiment, the detector array is configured to cover a region of biomagnetic interest, such as the torso or heart. In one such preferred embodiment, where the magnetometer is used as a cardiac magnetometer to detect and analyse the magnetic field of a subject's heart, the or each array comprises a hexagonal array of at least 7, e.g. 7-500 (or more), preferably at least 16, e.g. 16-500 (or more) detectors.

An increased number of detectors may be provided, e.g. where it is desired to measure the time-varying magnetic field of a subject's heart with a higher resolution and/or where it is desired to measure the time-varying magnetic field of a region of a subject's body other than the heart, such as in particular the brain. According to various preferred embodiments, the or each array may comprise a hexagonal array of 7, 19, 37, 61, 91, 127, 169, 217, 271, 331, 397 (or more) detectors.

The magnetometer system may comprise a single layer of detectors, or may comprise plural layers of one or more detectors, e.g. and preferably 2-10 (or more) layers, i.e. one above the other.

In one such embodiment, each detector layer comprises a single detector. In this case, then again, the magnetometer may be positioned appropriately over a subject (e.g. a subject's chest or other region of the subject's body) to take readings from a suitable (single) sampling position for the region of the subject's body in question. Alternatively, the magnetometer may be moved over the subject (e.g. the subject's chest) to take readings from plural different sampling positions in use. However, in a preferred embodiment, one or more or all of the detector layers comprise plural detectors, e.g. arranged in a two dimensional array, with one or more or each array preferably arranged as discussed above for the two dimensional array arrangement.

In these embodiments, one or more or each detector in each detector layer may be aligned with one or more or each detector in one or more or all of the other layers or otherwise (e.g. anti-aligned), as desired.

Where the magnetometer system comprises plural detectors, some or all of the detectors may be connected, e.g. in parallel and/or in series. Connecting plural detectors in series will have the effect of increasing the induced voltage for a given magnetic field strength. Connecting plural detectors in parallel will have the effect of reducing the thermal noise (Johnson noise) in the detectors. Preferably, a combination of series and parallel connections is used to optimise the balance of voltage and noise performance of the detectors.

In an embodiment, one or more or each detector in the magnetometer system is arranged in a gradiometer configuration, i.e. where two detectors are co-axially aligned (in the direction orthogonal to the plane in which each coil's windings are arranged), and where the signal from each of the coils is summed, e.g. to provide a measure of a change in the magnetic field in space.

The or each detector in the magnetometer system may comprise any suitable detector for detecting a time varying magnetic field.

The or each detector is preferably configured to be sensitive at least to magnetic signals between 0.1 Hz and 1 kHz, as this is the frequency range of the (majority of the) relevant magnetic signals of the heart. The or each detector may be sensitive magnetic signals outside of this range. The or each detector is preferably sensitive to magnetic fields in the range 10 fT-100 pT.

In a preferred embodiment, each detector in the magnetometer system comprises an induction coil. Thus, an induction coil or coils (i.e. a coil that is joined to an amplifier at both ends) is preferably used to detect the magnetic field of the subject (e.g. of the subject's heart). In this case, each coil may be configured as desired.

Each coil preferably has a maximum outer diameter less than 10 cm, preferably less than 7 cm, preferably between 4 and 7 cm. By limiting the outer diameter of the coil to 10 cm or less, a coil having an overall size that can achieve a spatial resolution that is suitable for medical magnetometry (and in particular for magneto cardiography) is provided. In particular, this facilitates a medically applicable diagnostic using 16 to 50 (or more) sampling positions (detection channels) to generate an image. (As discussed above, and as will be appreciated by those skilled in the art, the data for each sampling position can, e.g., be collected either by using an array of coils, or by using one (or several) coils that are moved around the chest to collect the data.) In a preferred embodiment, coils of around 7 cm diameter are used.

Each coil may have a non-magnetically active core (i.e. the coil windings may be wound around a non-magnetically active core), such as being air cored. Additionally or alternatively, one or more or each coil may comprise a magnetically active, such as ferrite or other magnetic material, core.

In one preferred embodiment, each coil corresponds to the arrangement described in the Applicants' earlier application WO2014/006387. Such coils can be used to provide a medical magnetometer that can be portable, relatively inexpensive, usable at room temperature and without the need for magnetic shielding, and yet can still provide sufficient sensitivity, accuracy and resolution to be medically useful.

However, the or each coil need not comprise the optimised coil in accordance with WO2014/006387, and may have any suitable and desired configuration.

The signal or signals that is or are output by the or each detector will (and preferably do) include a periodic (or pseudo-periodic) signal produced by the detector due to the time varying magnetic field of the region of the subject's body (together with noise).

The periodic (or pseudo-periodic) signal produced by the or each detector due to the time varying magnetic field of the region of the subject's body may include one or plural (different) signal features, i.e. one or plural (different) attributes or parts of the signal (that may or may not be of interest). For example, in the case of a signal produced due to the time varying magnetic field of a subject's heart, the signal may include (signal features corresponding to) the P wave, the QRS wave and/or the T wave, but may include other signal features.

In a preferred embodiment, the signal or signals from the one or more detectors is or are digitised, e.g. using one or more digitisers.

The or each digitiser may comprise any suitable digitiser that is operable to digitise (convert) an analogue signal received from the one or more detectors into a digital signal, e.g. for further processing and averaging, etc. The digitiser should (and preferably does) convert a voltage or current generated in the one or more detectors (coils) by the magnetic field into a digital signal.

In a preferred embodiment, the or each digitiser comprises an analogue to digital converter (ADC).

In a preferred embodiment, the magnetometer system comprises a digitiser coupled to each detector (each coil) and configured to digitise a signal from the detector. Where the system includes plural detectors, each detector may have its own, respective and separate, digitiser (i.e. there will be as many digitisers as there are detectors), or some or all of the detectors may share a digitiser.

The or each digitiser may be directly connected to the or each respective detector, or more preferably, the or each digitiser may be connected to the or each respective detector via an amplifier. Thus in a preferred embodiment, the magnetometer system includes one or more detection amplifiers, preferably in the form of a microphone amplifier (a low impedance amplifier), connected to one or more or each detector, e.g. to the ends of each coil. The or each detection amplifier is preferably then connected to a digitiser or digitisers.

The or each amplifier may be configured to have any suitable and desired amplification level. The or each amplifier may, for example, amplify the signal (including the noise) received from the or each detector by around 1000 times (60 dB) or more.

In a preferred embodiment, the magnetometer system is arranged such that the detector (e.g. coil) and amplifier (that is coupled to the detector (coil)) are arranged together in a sensor head or probe which is then joined by a wire to the remaining components of the magnetometer system to allow the sensor head (probe) to be spaced from the remainder of the magnetometer system in use.

Thus, in a preferred embodiment, the signal or signals that is filtered comprises a signal or signals from the one or more detectors that has been digitised, i.e. a digitised signal or signals from the one or more detectors.

In a preferred embodiment, the (preferably digitised) signal or signals from the one or more detectors, are averaged over plural periods, e.g. using averaging circuitry and/or software.

The digitised signal or signals may be averaged over plural periods as desired, and the averaging circuitry and/or software may comprise any suitable and desired circuitry and/or software for averaging the digitised signal or signals over plural periods.

In a preferred embodiment, a trigger is provided and used for gating (windowing) the signal (i.e. for identifying and dividing the periodic (or pseudo-periodic) signal into its plural repeating periods). The trigger should be, and preferably is, synchronised with the time varying magnetic field of the region of the subject's body. For example, where the magnetometer is used to analyse the magnetic field of a subject's heart, then the signal is preferably averaged over a number of heart beats, and an ECG or Pulse Ox trigger from the test subject may be used as a detection trigger for the signal acquisition process.

Thus, in a preferred embodiment, a trigger is used to identify each repeating period of the periodic (or pseudo-periodic) signal, and then the signal is averaged over the plural identified periods.

Other arrangements would, of course, be possible. For example, each repeating period of the (periodic or pseudo-periodic) signal may be identified without the use of a trigger, and then the signal may be averaged over the plural identified periods.

In the present invention, the (preferably digitised) signal or signals from the one or more detectors are filtered using a filter or filters. The filter or filters should be configured to attenuate (e.g. to remove) ballistocardiographic noise in the signal or signals, but may otherwise be configured in any suitable manner.

It would be possible to perform the filtering before signal averaging. Thus, in one embodiment, the signal or signals from the one or more detectors that are filtered comprise (non-averaged) signal or signals (directly) from the one or more detectors (or (directly) from the digitiser). However, in a preferred embodiment, the filtering is performed after signal averaging. As such, in a preferred embodiment, the signal or signals that are filtered comprise the averaged signal or signals.

The filter or filters should be (and are preferably) configured to filter the signal or signals from the one or more detectors so as to produce a filtered signal or signals.

In one embodiment, the attenuated part of the signal or signals is discarded (i.e. not used). Thus, in an embodiment, the filter or filters is configured to filter the signal or signals from the one or more detectors so as to remove (and discard) the ballistocardiographic noise.

However, it would also be possible to retain the ballistocardiographic (the attenuated (removed)) part of the signal or signals, and to use it for some other purpose, e.g. as a diagnostic indicator. Thus, in an embodiment, the filter or filters is configured to filter the signal or signals from the one or more detectors so as to produce both (e.g. to separate out) the filtered signal or signals and one or more other (e.g. ballistocardiographic noise) signals.

It would also be possible, in a mode of operation, to retain (and use) only the ballistocardiographic part of the signal or signals.

The filter or filters is configured to attenuate ballistocardiographic noise in the signal or signals, i.e. so as to produce the filtered signal or signals.

In this regard, “ballistocardiographic noise” comprises magnetic noise detected by a detector that is caused by motion (vibration) of a support structure that comprises electrically conductive, e.g. metallic, ferrous and/or magnetic material and that is supporting a subject's body, where the motion is correlated (synchronised) with (e.g. caused by) motion of the region of the subject's body in question (e.g. the subject's heartbeat). In particular, “ballistocardiographic noise” refers to magnetic noise detected by a detector that is caused by motion (vibration) of the support structure, where the motion is correlated (synchronised) with (e.g. caused by) the recoil forces of the body in reaction to cardiac ejection of blood into the vasculature.

The Applicants have furthermore recognised that other types of magnetic noise of biological origin, i.e. that is caused by motion (vibration) of the support structure (e.g. bed) (that comprises electrically conductive, e.g. metallic, ferrous and/or magnetic material), which motion is correlated (synchronised) with (e.g. caused by) motion of the region of the subject's body in question, exist and can be attenuated using the filter or filters of the present invention.

For example, while ballistocardiographic noise may be due to the recoil forces of the body in reaction to cardiac ejection of blood into the vasculature, “seismocardiographic noise” may be due to local vibrations of the chest wall in response to the heartbeat.

Other sources of synchronous biological noise include, for example, breathing (e.g. where the region of the subject's body in question comprises the abdomen, chest or lung(s)).

In addition, changes in the position of the subject's body on the support structure, e.g. due to talking, fidgeting, etc., can cause motion (vibration) of the support structure which in turn can produce magnetic noise in the signal or signals. Such noise may be synchronised with motion of the region of the subject's body in question, e.g. where the region of the subject's body in question comprises one or more muscles.

These biological sources of synchronous noise should be contrasted with e.g. other sources of noise such as nearby vibrations (e.g. vibrating lift shafts, large objects being dropped or moved, etc.). Although these other noise sources can cause motion (vibration) of the support structure which in turn can produce magnetic noise in the signal or signals (which noise may appear to be similar to the synchronous biological noise), generally such noise is not synchronised with the motion of region of the subject's body in question (e.g. heartbeat) and can therefore be reduced using averaging (over a long enough period of time). (It should be noted, however, that the filter or filters of the present invention may also remove some or all of this non-synchronous noise, i.e. in addition to the synchronous biological noise described above.)

Thus, according to another aspect of the present invention, there is provided a method of using a magnetometer system to analyse the magnetic field of a region of a subject's body, the method comprising:

using one or more detectors to detect the time varying magnetic field of a region of a subject's body;

filtering a signal or signals from the one or more detectors using a filter or filters, wherein the filter or filters is configured to attenuate noise in the signal or signals that is synchronised with motion of the region of the subject's body; and

using the filtered signal or signals to analyse the magnetic field generated by the region of a subject's body.

According to another aspect of the present invention, there is provided a magnetometer system for medical use, comprising:

one or more detectors for detecting the time varying magnetic field of a region of a subject's body; and

a filter or filters configured to filter a signal or signals from the one or more detectors, wherein the filter or filters is configured to attenuate noise in the signal or signals that is synchronised with motion of the region of the subject's body;

wherein the magnetometer system is configured to provide the filtered signal or signals for use to analyse the magnetic field generated by the region of the subject's body.

As will be appreciated by those skilled in the art, these aspects of the present invention can and preferably do include any one or more or all of the preferred and optional features of the invention described herein, as appropriate.

Attenuating the synchronised, e.g. ballistocardiographic noise should (and preferably does) comprise reducing the amplitude of the synchronised, e.g. ballistocardiographic noise (e.g. at least in the filtered signal or signals). More preferably, attenuating the synchronised, e.g. ballistocardiographic noise comprises (completely) removing the synchronised, e.g. ballistocardiographic noise (e.g. at least from the filtered signal or signals).

The filter or filters should be (and is preferably) configured to attenuate (e.g. separate or remove) the synchronised, e.g. ballistocardiographic noise in the signal or signals without attenuating (or attenuating to a lesser degree), and preferably without (significantly) distorting, some or all of the “useful”, wanted, part of the signal.

In this regard, the conventional approach to analysing the magnetic field of a subject's heart is to keep as much of the signal originating from the heart as possible. As described above, this will include the P wave, the QRS wave and/or the T wave. Thus, conventionally, care is taken to retain as much of the P wave, the QRS wave and the T wave in the signal as possible. As also described above, the Applicants have found that the ballistocardiographic noise appears in a frequency range that overlaps with the frequency range of this conventionally “wanted” signal.

However, the Applicants have furthermore recognised that the QRS complex is particularly important in terms of providing diagnostic information, and that the T-wave is less important in this regard. The Applicants have also recognised that the ballistocardiographic noise appears (mainly) in a frequency range that overlaps with the frequency range of the T-wave. This means that the filter can be (and is preferably) configured to attenuate (e.g. separate or remove) the ballistocardiographic noise (together with the T-wave) in the signal or signals without attenuating (or attenuating to a lesser degree), and preferably without (significantly) distorting, the “useful”, wanted, QRS complex.

Thus, the filter or filters is preferably configured to allow at least the QRS complex to pass (preferably without being attenuated and/or distorted) and to attenuate (e.g. to separate or remove) the synchronised, e.g. ballistocardiographic noise, i.e. so as to produce the filtered signal or signals. Filtering the signal or signals in this manner allows the synchronised, e.g. ballistocardiographic noise to be removed from the signal, without (significantly) affecting the medically useful QRS complex.

In this regard, the Applicants have recognised that the ballistocardiographic bed noise comprises (mainly) lower frequency components, e.g. when compared with the frequency range at which the QRS complex appears. Thus, the filter is preferably configured to allow at least the QRS complex to pass (preferably without being attenuated and/or distorted) and to attenuate (e.g. to separate or remove) parts of the signal having frequencies less than the frequency range at which the QRS complex appears.

In a preferred embodiment, the filter is configured to attenuate (e.g. to separate or remove) signal or signals having frequencies below a particular, preferably selected, cut-off frequency (threshold) (i.e. the filter is configured to attenuate components of the signal or signals with frequencies below the cut-off frequency). The filter may be configured to attenuate (e.g. to separate or remove) only some frequencies less than the cut-off frequency, but more preferably the filter is configured to attenuate (e.g. to separate or remove) all frequencies less than the cut-off frequency.

Thus, in a preferred embodiment, the or each filter comprises a high-pass filter, i.e. where the high-pass filter has a low frequency cut-off (i.e. a frequency (threshold) below which (most of) the signal is attenuated (but above which (most of) the signal is passed by the high-pass filter)), and filtering the signal or signals comprises high-pass filtering the signal or signals.

Correspondingly, according to another aspect of the present invention, there is provided a method of using a magnetometer system to analyse the magnetic field of a region of a subject's body, the method comprising:

using one or more detectors to detect the time varying magnetic field of a region of a subject's body;

filtering a signal or signals from the one or more detectors using a filter or filters, wherein the filter or filters comprises a high-pass filter; and

using the filtered signal or signals to analyse the magnetic field generated by the region of a subject's body.

According to another aspect of the present invention, there is provided a magnetometer system for medical use, comprising:

one or more detectors for detecting the time varying magnetic field of a region of a subject's body; and

a filter or filters configured to filter a signal or signals from the one or more detectors, wherein the filter or filters comprises a high-pass filter;

wherein the magnetometer system is configured to provide the filtered signal or signals for use to analyse the magnetic field generated by the region of the subject's body.

As will be appreciated by those skilled in the art, these aspects of the present invention can and preferably do include any one or more or all of the preferred and optional features of the invention described herein, as appropriate.

Thus, for example, the filter or filters may be configured to filter the signal or signals from the one or more detectors so as to produce only the filtered signal or signals (i.e. where the attenuated, low frequency, part of the signal or signals is discarded), or the filter or filters may be configured to filter the signal or signals from the one or more detectors so as to produce both (e.g. to separate out) the filtered signal or signals and one or more other (e.g. low frequency) signals (e.g. where the low frequency part of the signal or signals is retained and used), e.g. as described above.

The or each high-pass filter may be configured in any suitable manner. In a particularly preferred embodiment, the high-pass filter comprises a windowed sinc filter. This is a particularly beneficial arrangement since the windowed sinc filter can provide a good approximation to the ideal “brick wall” high-pass filter.

The low frequency cut-off may be selected as desired. However, in a preferred embodiment, the filter has a low frequency cut-off between around 8 and 12 Hz, more preferably between around 9 and 11 Hz. Most preferably, the filter is configured to have a low frequency cut-off at around 10 Hz.

In this regard, the Applicants have found in particular that the ballistocardiographic noise or “bed noise” appears in the frequency range around <10 Hz, whereas the T-wave appears in the frequency range around 4-7 Hz and the QRS complex appears at frequencies >10 Hz. Accordingly, the use of a low frequency cut-off at around 10 Hz results in removal of a significant proportion of the ballistocardiographic noise from the signal or signals, without significantly affecting the medically useful part of the signal or signals.

The filter or filters is preferably configured to have a relatively narrow roll-off. Again, this means that the filter will function as close as possible to the ideal “brick wall” filter.

In this regard, the Applicants have recognised that configuring the filter in this manner will have the effect of increasing the pass band and/or stop band ripple, but that the shape of the roll off is more important in the present invention, where it is desired to remove synchronised, e.g. ballistocardiographic noise or “bed noise” from the signal. This is because the ballistocardiographic noise or “bed noise” appears adjacent in frequency to the useful QRS complex part of the signal.

Thus, according to another aspect of the present invention, there is provided a method of using a magnetometer system to analyse the magnetic field of a region of a subject's body, the method comprising:

using one or more detectors to detect the time varying magnetic field of a region of a subject's body;

filtering a signal or signals from the one or more detectors using a filter or filters, wherein the filter or filters is configured to attenuate signals having frequencies less than a low frequency cut-off frequency, wherein the low frequency cut-off frequency is between around 8 and 12 Hz; and

using the filtered signal or signals to analyse the magnetic field generated by the region of a subject's body.

According to another aspect of the present invention, there is provided a magnetometer system for medical use, comprising:

one or more detectors for detecting the time varying magnetic field of a region of a subject's body; and

a filter or filters configured to filter a signal or signals from the one or more detectors, wherein the filter or filters is configured to attenuate signals having frequencies less than a low frequency cut-off frequency, wherein the low frequency cut-off frequency is between around 8 and 12 Hz;

wherein the magnetometer system is configured to provide the filtered signal or signals for use to analyse the magnetic field generated by the region of the subject's body.

As will be appreciated by those skilled in the art, these aspects of the present invention can and preferably do include any one or more or all of the preferred and optional features of the invention described herein, as appropriate.

Thus, for example, the filter or filters may be configured to filter the signal or signals from the one or more detectors so as to produce only the filtered signal or signals (i.e. where the attenuated, low frequency, part of the signal or signals is discarded), or the filter or filters may be configured to filter the signal or signals from the one or more detectors so as to produce both (e.g. to separate out) the filtered signal or signals and one or more other (e.g. low frequency) signals (e.g. where the attenuated (removed), low frequency, part of the signal or signals is retained and used), e.g. as described above.

In a particularly preferred embodiment, the filter or filters is additionally configured to attenuate (e.g. to separate or remove) other (high-frequency) background noise in the signal or signals. As such, a single filter may be (and is preferably) used to attenuate multiple types of noise in the signal or signals.

In these embodiments, the or each filter should be (and is preferably) configured to attenuate the other (high-frequency) background noise in the signal or signals without attenuating (or attenuating to a lesser degree), and preferably without (significantly) distorting, at least some of the “useful”, wanted, part of the signal. Thus, the filter is preferably configured to allow at least the QRS complex to pass (preferably without being attenuated and/or distorted) and to attenuate (e.g. to separate or remove) the other (high-frequency) background noise.

In this regard, the Applicants have recognised that other background noise that has (mainly) relatively high frequency components (e.g. when compared with the frequency range at which the QRS complex appears), such as mains power noise, may be present in the signal or signals. Thus, the filter is preferably configured to allow at least the QRS complex to pass (preferably without being attenuated and/or distorted) and to attenuate (e.g. to separate or remove) parts of the signal having frequencies greater than the frequency range at which the QRS complex appears.

In a preferred embodiment, the filter or filters is configured to attenuate (e.g. to separate or remove) signal or signals having frequencies higher than a particular, preferably selected, high frequency cut-off frequency (threshold) (i.e. the filter is configured to attenuate components of the signal or signals with frequencies above the high frequency cut-off frequency). The filter may be configured to attenuate only some frequencies higher than the high frequency cut-off frequency, but more preferably the filter is configured to attenuate all frequencies higher than the high frequency cut-off frequency.

Thus, in a preferred embodiment, the filter or filters comprises a low-pass filter, i.e. where the low-pass filter has a high frequency cut-off (i.e. a frequency (threshold) above which (most of) the signal is attenuated (but below which (most of) the signal is passed by the low-pass filter)), and filtering the signal or signals comprises low-pass filtering the signal or signals.

The low-pass filter may be configured in any suitable manner. In a particularly preferred embodiment, the low-pass filter comprises a windowed sinc filter.

The high frequency cut-off may be selected as desired.

In this regard, the Applicants have found, in particular that the other (high-frequency) background noise, in particular environmental noise such as mains power noise, appears in the frequency range around 50 Hz, whereas the QRS complex appears at frequencies <50 Hz, and accordingly that the use of a high frequency cut-off at around 50 Hz (and preferably less than this) results in removal of a significant proportion of the other (high-frequency) background noise from the signal or signals, without significantly affecting the medically useful part of the signal or signals.

Thus, in a preferred embodiment, the filter has a high frequency cut-off at or below around 50 Hz, preferably between around 45 and 50 Hz, more preferably between around 45 and 48 Hz.

Where the mains power noise appears at another frequency, e.g. at around 60 Hz, then the filter may be configured to have a high frequency cut-off at or below that other frequency. Thus, in a preferred embodiment, the filter has a high frequency cut-off at or below around 60 Hz, preferably between around 55 and 60 Hz, more preferably between around 55 and 58 Hz.

It will accordingly be appreciated that in a particularly preferred embodiment, the filter is configured to attenuate (e.g. to separate or remove) synchronised, e.g. ballistocardiographic noise and other (high-frequency) background noise in the signal or signals, preferably without attenuating (or attenuating to a lesser degree), and preferably without (significantly) distorting, the “useful”, wanted, part of the signal, i.e. the QRS complex.

In a preferred embodiment, the filter is configured to allow at least the QRS complex to pass (preferably without being attenuated and/or distorted) and to attenuate (e.g. to separate or remove) parts of the signal having frequencies outside the frequency range at which the QRS complex appears.

In a preferred embodiment, the filter or filters is configured to attenuate (e.g. to separate or remove) signal or signals having frequencies below a particular, preferably selected, low frequency cut-off (threshold) and to attenuate (e.g. to separate or remove) signal or signals having frequencies above a particular, preferably selected, high frequency cut-off (threshold). Thus, the filter or filters is preferably configured to attenuate signal or signals having frequencies outside a particular, preferably selected, frequency range.

The filter may be configured to attenuate (e.g. to separate or remove) only some frequencies higher than the high frequency cut-off and only some frequencies less than the low frequency cut-off, but more preferably the filter is configured to attenuate (e.g. to separate or remove) all frequencies higher than the high frequency cut-off and all frequencies less than the low frequency cut-off.

Thus, in a preferred embodiment, the filter or filters comprises a band-pass filter, i.e. where the band-pass filter has a low frequency cut-off (threshold) and a high frequency cut-off (threshold), and filtering the signal or signals comprises band-pass filtering the signal or signals, i.e. so as to produce the filtered signal or signals.

Correspondingly, according to another aspect of the present invention, there is provided a method of using a magnetometer system to analyse the magnetic field of a region of a subject's body, the method comprising:

using one or more detectors to detect the time varying magnetic field of a region of a subject's body;

filtering a signal or signals from the one or more detectors using a filter or filters, wherein the filter or filters comprises a band-pass filter; and

using the filtered signal or signals to analyse the magnetic field generated by the region of a subject's body.

According to another aspect of the present invention, there is provided a magnetometer system for medical use, comprising:

one or more detectors for detecting the time varying magnetic field of a region of a subject's body; and

a filter or filters configured to filter a signal or signals from the one or more detectors, wherein the filter or filters comprises a band-pass filter;

wherein the magnetometer system is configured to provide the filtered signal or signals for use to analyse the magnetic field generated by the region of the subject's body.

As will be appreciated by those skilled in the art, these aspects of the present invention can and preferably do include any one or more or all of the preferred and optional features of the invention described herein, as appropriate.

The or each band-pass filter may be configured in any suitable manner. In a particularly preferred embodiment, the band-pass filter comprises a combination of (i.e. the difference between) two windowed sinc filters.

The windowed sinc filter or filters should be (and preferably are) configured to have a particular, preferably selected, window function. The filter window function or functions may be selected as desired. Suitable window functions include, for example, the Hamming window, the Blackman window, the Bartlett window, the Hanning window, etc.

In a particularly preferred embodiment, the or each windowed sinc filter uses a Blackman window. The Applicants have found that the Blackman window is particularly suited for use in preferred embodiments of the present invention. Although the Blackman window has a slower roll-off compared with the other types of window function (e.g. the Hamming window), it has an improved stopband attenuation, and a lower passband ripple.

Similarly, the or each windowed sinc filter should (and preferably does) have a particular, preferably selected, filter kernel length, M. In the frequency domain, the length of the filter kernel M determines the transition bandwidth of the filter, BW. There is a trade-off between computation time (which depends on the value of M) and the filter sharpness (the value of BW), which can be expressed through the approximation:

$$M \approx \frac{4}{BW} .$$

As such, the sharper the filter is (the smaller the transition bandwidth BW), the longer is the time required to perform convolution in the time domain.

The filter is preferably configured to have a relatively narrow roll-off. Again, this means that the filter will function as close as possible to the ideal “brick wall” filter.

In a particularly preferred embodiment, the length of the filter kernel, M is set to be equal to one second, i.e. of averaged signal (and therefore to be equal to the sampling rate). This minimises the transition bandwidth BW.

The passband of the band pass filter may be selected as desired. However, in a preferred embodiment, the passband has a low frequency cut-off between around 8 and 12 Hz, and a high frequency cut-off between around 45 and 50 Hz, more preferably between around 45 and 48 Hz. It would also be possible for the high frequency cut-off to be between around 55 and 60 Hz, more preferably between around 55 and 58 Hz, e.g. as described above. Most preferably, the filter is configured to have a passband at around 10 to 50 Hz.

The Applicants have found that this arrangement provides a practical and efficient way to examine the signal and extract the “useful” MCG features reliably, especially in a noisy environment such as an emergency department. However, other arrangements would be possible.

Thus, according to another aspect of the present invention, there is provided a method of using a magnetometer system to analyse the magnetic field of a region of a subject's body, the method comprising:

using one or more detectors to detect the time varying magnetic field of a region of a subject's body;

filtering a signal or signals from the one or more detectors using a filter or filters, wherein the filter or filters is configured to attenuate signals having frequencies less than a low frequency cut-off frequency, wherein the low frequency cut-off frequency is between around 8 and 12 Hz and to attenuate signals having frequencies greater than a high frequency cut-off frequency, wherein the high frequency cut-off frequency is between around 45 and 60 Hz; and

using the filtered signal or signals to analyse the magnetic field generated by the region of a subject's body.

According to another aspect of the present invention, there is provided a magnetometer system for medical use, comprising:

one or more detectors for detecting the time varying magnetic field of a region of a subject's body; and

a filter or filters configured to filter a signal or signals from the one or more detectors, wherein the filter or filters is configured to attenuate signals having frequencies less than a low frequency cut-off frequency, wherein the low frequency cut-off frequency is between around 8 and 12 Hz and to attenuate signals having frequencies greater than a high frequency cut-off frequency, wherein the high frequency cut-off frequency is between around 45 and 60 Hz;

wherein the magnetometer system is configured to provide the filtered signal or signals for use to analyse the magnetic field generated by the region of the subject's body.

As will be appreciated by those skilled in the art, these aspects of the present invention can and preferably do include any one or more or all of the preferred and optional features of the invention described herein, as appropriate.

Thus, for example, the filter or filters may be configured to filter the signal or signals from the one or more detectors so as to produce only the filtered signal or signals (i.e. where the attenuated parts of the signal or signals are discarded), or the filter or filters may be configured to filter the signal or signals from the one or more detectors so as to produce both (e.g. to separate out) the filtered signal or signals and one or more other (e.g. low and/or high frequency) signals (e.g. where the low frequency parts of the signal or signals are retained and used), e.g. as described above.

In the present invention, the filtered signal or signals is used to analyse the magnetic field generated by the region of the subject's body. This may be done in any suitable manner.

A heartbeat's waveform and/or information such as a time interval or intervals e.g. between separate heartbeats and/or between certain features within a single heartbeat, and/or a shape or shapes of a heartbeat(s) may be obtained from the filtered signal or signals.

In one preferred embodiment, the filtered signal or signals are subjected to appropriate signal processing, for example to generate false colour images of the magnetic field or otherwise.

Thus, in a preferred embodiment, the filtered signal or signals are used to provide an output indicative of the time varying magnetic field. This preferably comprises providing a display indicative of the time varying magnetic field, e.g. displaying an image indicative of the time varying magnetic field on a display. Most preferably, the filtered signal or signals are used to provide a false colour image or images indicative of the time varying magnetic field, and the false colour image or images are displayed on a display.

In a preferred embodiment, suitable measurements are taken to allow an appropriate magnetic scan image of the heart (or other body region of interest) to be generated, which image can then, e.g., be compared to reference images for diagnosis. The present invention can be used to carry out any known and suitable procedure for imaging the magnetic field of the heart.

Preferably plural (e.g. 7 to 500 (or more), as described above) sampling positions (detection channels) are detected in order to generate the desired scan image.

The present invention accordingly extends to the use of the magnetometer system of the present invention for analysing, and preferably for imaging, the magnetic field generated by a subject's heart (or other body region), and to a method of analysing, and preferably of imaging, the magnetic field generated by a subject's heart (or other body region) comprising using the method or system of the present invention to analyse, and preferably to image, the magnetic field generated by a subject's heart (or other region of the body). The analysis, and preferably the generated information and/or image, is preferably used for diagnosis of (to diagnose) a medical condition, such as abnormality of the heart, etc.

Thus according to another aspect of the present invention, there is provided a method of diagnosing a medical condition, comprising:

using one or more detectors to detect the time varying magnetic field of a region of a subject's body;

filtering a signal or signals from the one or more detectors using a filter or filters, wherein the filter or filters is configured to attenuate synchronised, e.g. ballistocardiographic noise in the signal or signals;

using the filtered signal or signals to analyse the magnetic field generated by the region of a subject's body; and

using the analysis of the magnetic field generated by the region of the subject's body to diagnose said medical condition.

In this aspect of the present invention, the signal (features of interest) from the detector or detectors are preferably used to produce an image representative of the magnetic field generated by the region of the subject's body, and the method preferably then comprises comparing the image obtained with a reference image or images to diagnose the medical condition. The medical condition is, as discussed above, preferably one of: abnormality of the heart, a bladder condition, pre-term labour, foetal abnormalities or abnormality of the head or brain.

As will be appreciated by those skilled in the art, all of the aspects and embodiments of the invention described herein can and preferably do include any one or more or all of the preferred and optional features of the present invention, as appropriate.

The methods in accordance with the present invention may be implemented at least partially using software e.g. computer programs. It will thus be seen that when viewed from further aspects the present invention provides computer software specifically adapted to carry out the methods herein described when installed on data processing means, a computer program element comprising computer software code portions for performing the methods herein described when the program element is run on data processing means, and a computer program comprising code means adapted to perform all the steps of a method or of the methods herein described when the program is run on a data processing system. The data processing system may be a microprocessor, a programmable FPGA (Field Programmable Gate Array), etc.

The invention also extends to a computer software carrier comprising such software which when used to operate a magnetometer system comprising data processing means causes in conjunction with said data processing means said system to carry out the steps of the methods of the present invention. Such a computer software carrier could be a physical storage medium such as a ROM chip, CD ROM or disk, or could be a signal such as an electronic signal over wires, an optical signal or a radio signal such as to a satellite or the like.

It will further be appreciated that not all steps of the methods of the invention need be carried out by computer software and thus from a further broad aspect the present invention provides computer software and such software installed on a computer software carrier for carrying out at least one of the steps of the methods set out herein.

The present invention may accordingly suitably be embodied as a computer program product for use with a computer system. Such an implementation may comprise a series of computer readable instructions either fixed on a tangible medium, such as a non-transitory computer readable medium, for example, diskette, CD ROM, ROM, or hard disk. It could also comprise a series of computer readable instructions transmittable to a computer system, via a modem or other interface device, over either a tangible medium, including but not limited to optical or analogue communications lines, or intangibly using wireless techniques, including but not limited to microwave, infrared or other transmission techniques. The series of computer readable instructions embodies all or part of the functionality previously described herein.

Those skilled in the art will appreciate that such computer readable instructions can be written in a number of programming languages for use with many computer architectures or operating systems. Further, such instructions may be stored using any memory technology, present or future, including but not limited to, semiconductor, magnetic, or optical, or transmitted using any communications technology, present or future, including but not limited to optical, infrared, or microwave. It is contemplated that such a computer program product may be distributed as a removable medium with accompanying printed or electronic documentation, for example, shrink wrapped software, pre-loaded with a computer system, for example, on a system ROM or fixed disk, or distributed from a server or electronic bulletin board over a network, for example, the Internet or World Wide Web.

A number of preferred embodiments of the present invention will now be described by way of example only and with reference to the accompanying drawings, in which:

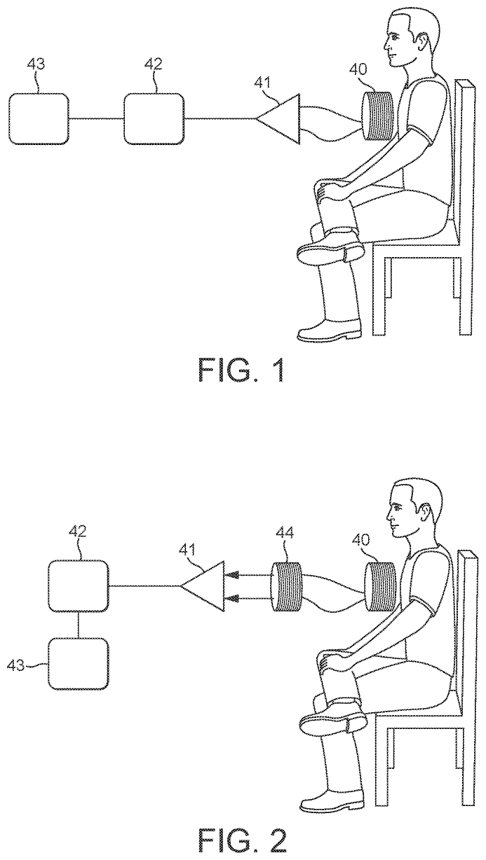

FIG. 1 shows schematically the use of an embodiment of the present invention for detecting the magnetic field of a subject's heart;

FIGS. 2-5 show further exemplary arrangements of the use of an embodiment of the present invention when detecting the magnetic field of a subject's heart;

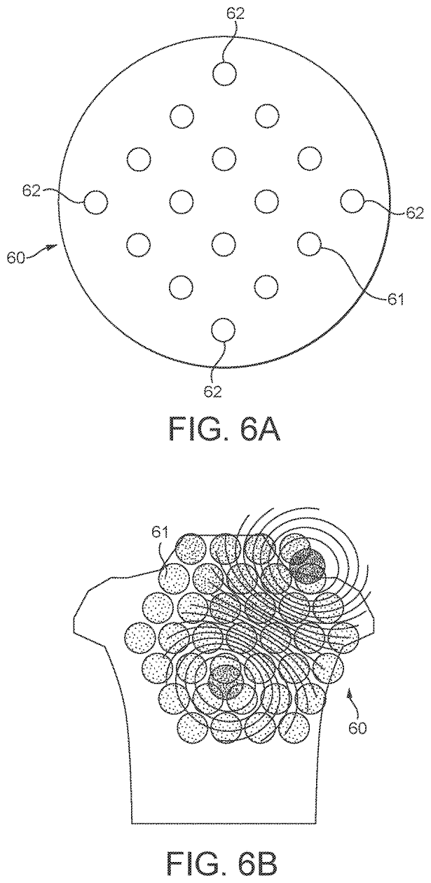

FIG. 6A shows schematically a coil arrangement in accordance with an embodiment of the present invention, and FIG. 6B shows schematically another coil arrangement in accordance with an embodiment of the present invention;

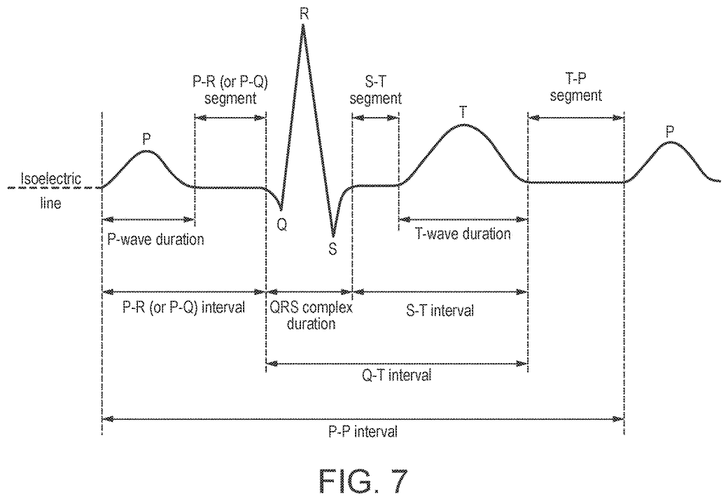

FIG. 7 shows a typical healthy ECG trace;

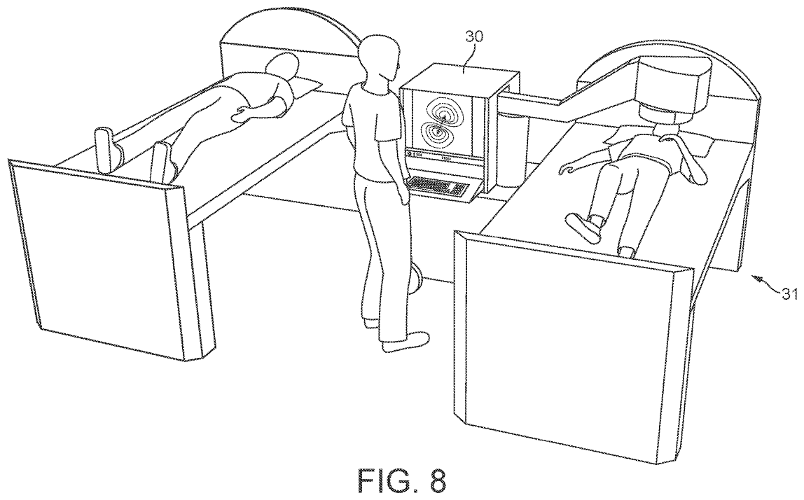

FIG. 8 shows a further exemplary arrangement of the use of an embodiment of the present invention when detecting the magnetic field of a subject's heart;

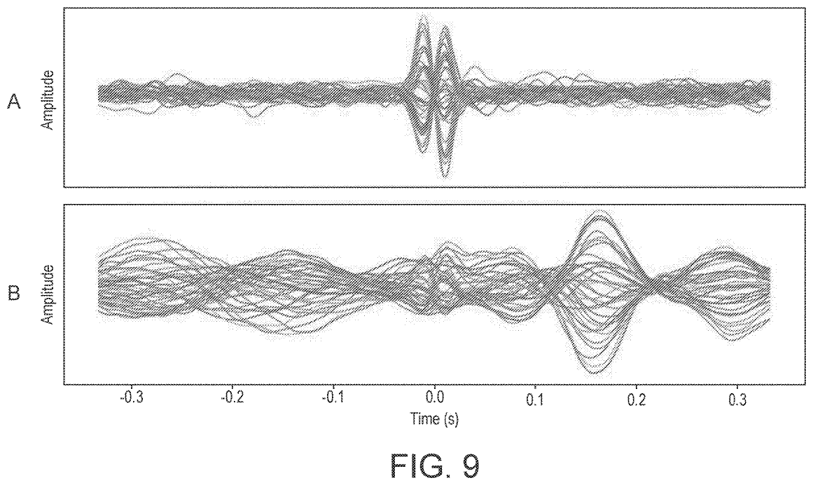

FIG. 9A shows cycle averaged MCG data for a healthy subject captured by a 37-channel magnetometer device in an unshielded environment on a wooden bed, and FIG. 9B shows cycle averaged MCG data for a healthy subject captured by a 37-channel magnetometer device in an unshielded environment on a bed comprising ferrous (magnetic) material;

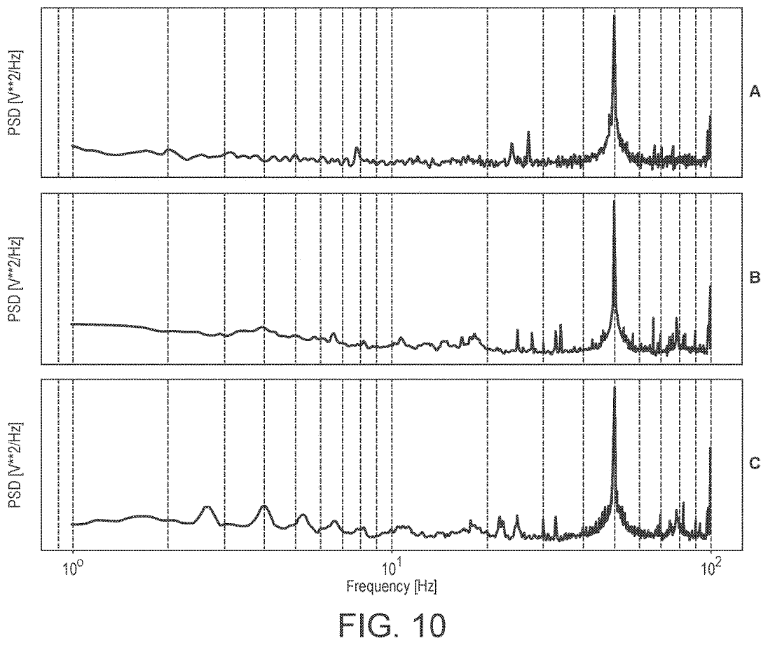

FIG. 10 A shows a log periodogram of MCG data captured by a 37-channel magnetometer device in an unshielded environment without a subject present, FIG. 10B shows a log periodogram of MCG data for a healthy subject captured by a 37-channel magnetometer device in an unshielded environment on a wooden bed, and FIG. 100 shows corresponding data for a bed comprising ferrous (magnetic) material;

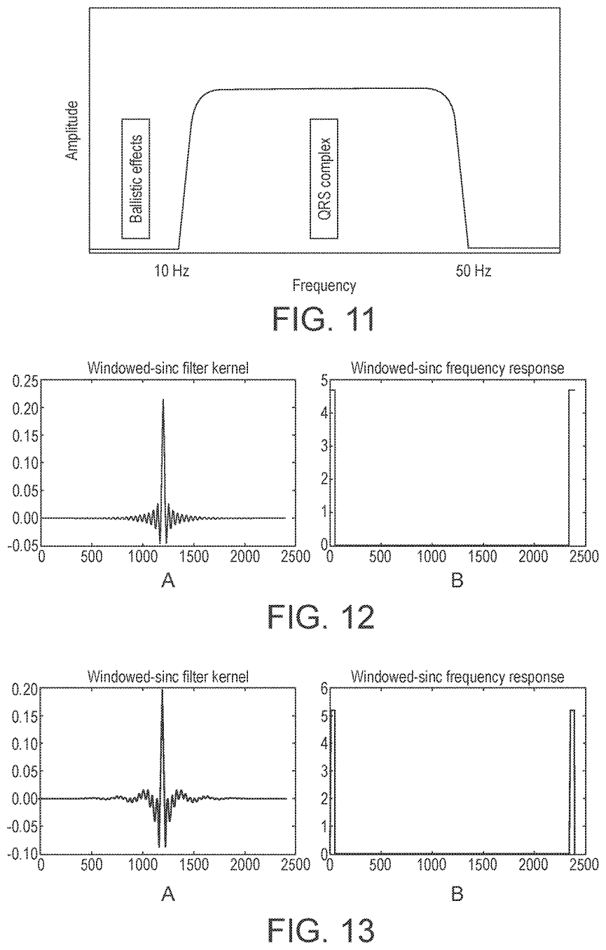

FIG. 11 illustrates an ideal band-pass filter in the frequency domain;

FIG. 12A shows a windowed-sinc filter kernel with a cut-off frequency at 45 Hz and M=2400, and FIG. 12B shows the frequency response of the filter;

FIG. 13A shows a filter kernel formed from the difference between two windowed-sinc filters with cut-off frequencies at 8 Hz and 45 Hz, and M=2400, and FIG. 13B shows the frequency response of the filter;

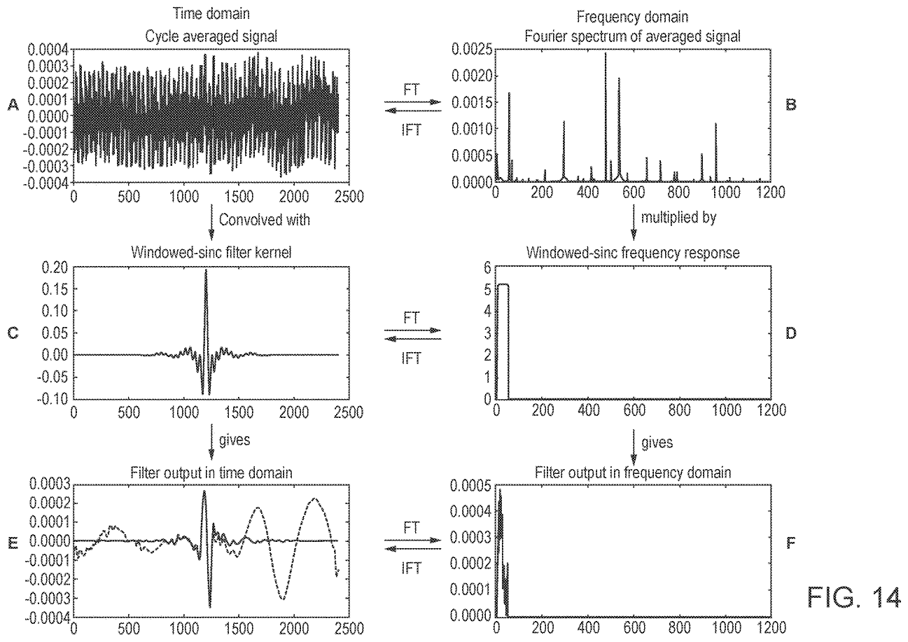

FIG. 14A shows an averaged MCG signal recorded in a non-shielded room, FIG. 14B shows the Fourier spectrum of the signal of FIG. 14A, FIG. 14C shows a windowed-sinc filter kernel with cut-off frequencies at 8 Hz and 45 Hz, FIG. 14D shows the corresponding frequency response of the filter kernel of FIG. 14C, FIG. 14E shows the result of applying the filter in the time domain to the signal of FIG. 14A (solid line) and the result of applying a filter with cut-off frequencies at 2 Hz and 45 Hz to the signal of FIG. 14A (dashed line), and FIG. 14F shows the result of applying the filter in the frequency domain to the signal of FIG. 14A;

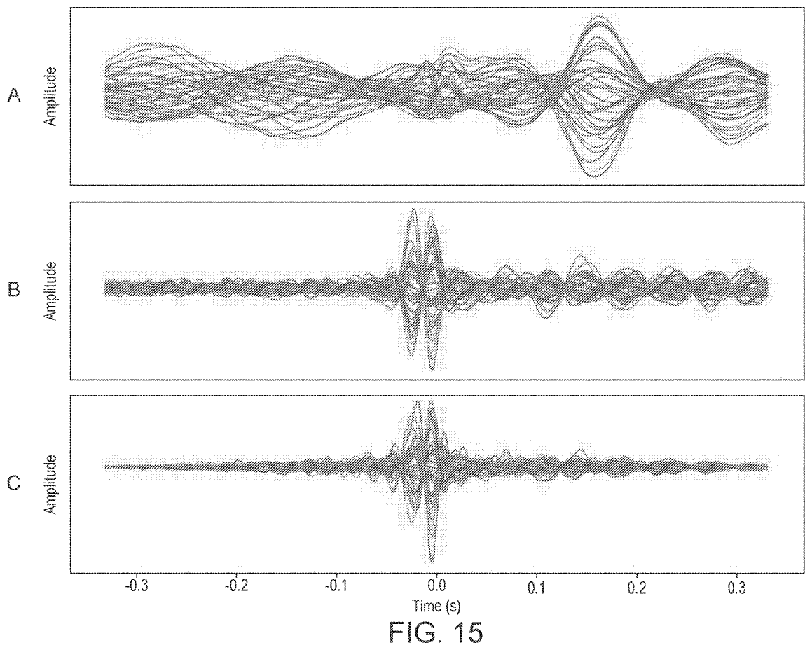

FIG. 15A again shows the cycle averaged MCG data for a healthy subject captured by a 37-channel magnetometer device in an unshielded environment on a bed comprising ferrous (magnetic) material of FIG. 9B, and FIGS. 15B and 15C show the data after a windowed sinc filter kernel with a Blackman window and cut-off frequencies at 8 Hz and 45 Hz has been applied to the data;

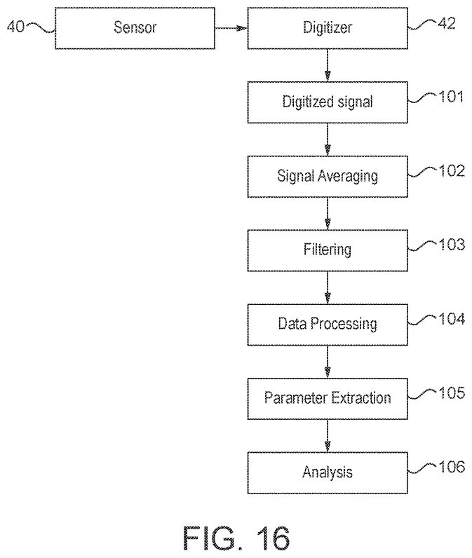

FIG. 16 illustrates a process in accordance with an embodiment of the present invention.

Like reference numerals are used for like components where appropriate in the Figures.

Magnetocardiography (MCG) is the measurement of magnetic fields emitted by the heart caused by the electrical current within myocardium heart cells. The measurement of these fields provides diagnostically significant information which is complimentary to that obtained by electrocardiography (ECG), and can be used to diagnose abnormalities of heart function.

FIG. 1 shows schematically the basic arrangement of a preferred embodiment of a magnetometer system that may be operated in accordance with the present invention. This magnetometer system is specifically intended for use as a cardiac magnetometer (for use to detect the magnetic field of a subject's heart). However, the same magnetometer design can be used to detect the magnetic field produced by other body regions, for example for detecting and diagnosing bladder conditions, pre-term labour, foetal abnormalities and for magnetoencephalography. Thus, although the present embodiment is described with particular reference to cardio-magnetometry, it should be noted that the present embodiment (and the present invention) extends to other medical uses as well.

The magnetometer system comprises a detector 40 coupled to a detection circuit 41 that may contain a number of components. The detector 40 may be an induction coil 40. The detection circuit 41 may comprise a low impedance preamplifier, such as a microphone amplifier, that is connected to the coil 40.

The current output from the coil 40 is processed and converted to a voltage by the detection circuit 41 and provided to an analogue to digital converter (ADC) 42 which digitises the analogue signal from the coil 40 and provides it to a data acquisition system 43.

A biological signal that is correlated to the heartbeat, e.g. an ECG or Pulse-Ox trigger from the test subject may be used as a detection trigger for the digital signal acquisition, and the digitised signal over a number of trigger pulses is then binned into appropriate signal bins, and the signal bins overlaid or averaged, by the data acquisition unit 43. Other arrangements would, however, be possible.

The coil 40 and detection circuit 41 may be arranged such that the coil 40 and the preamplifier of the detection circuit 41 are arranged together in a sensor head or probe which is then joined by a wire to a processing circuit that comprises the remaining components of the detection circuit 41. Connecting the sensor head (probe) and the processing circuit by wire allows the processing circuit to be spaced from the sensor head (probe) in use.

With this magnetometer, the sensor head (probe) will be used as a magnetic probe by placing it in the vicinity of the magnetic fields of interest.

FIG. 2 shows an improvement over the FIG. 1 arrangement, which uses in particular the technique of gradient subtraction to try to compensate for background noise. (Other techniques could, however, be used). In this case, an inverse coil 44 is used to attempt to subtract the effect of the background noise magnetic field from the signal detected by the probe coil 40. The inverse coil 44 will, as is known in the art, be equally sensitive to any background magnetic field, but only weakly sensitive to the subject's magnetic field. The inverse coil 44 can be accurately matched to the pickup coil 40 by, for example, using a movable laminated core to tune the performance of the inverse coil to that of the pickup coil 40.

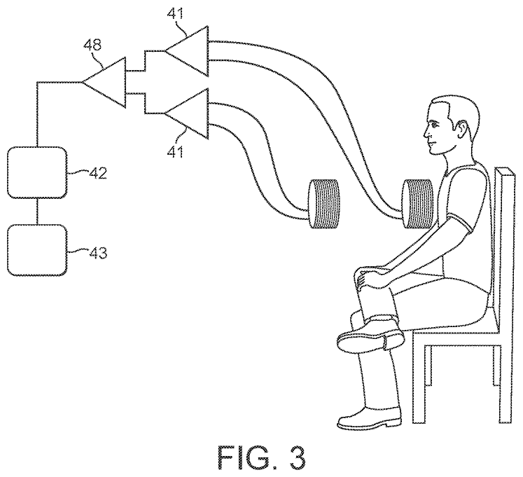

FIG. 3 shows an alternative gradient subtraction arrangement. In this case, both coils 40, 44 have the same orientation, but their respective signals are subtracted using a differential amplifier 45. Again, the best operation is achieved by accurately matching the coils and the performance of the detection circuits 41. Again, a movable laminated core can be used to tune the performance of one coil to match the performance of the other.

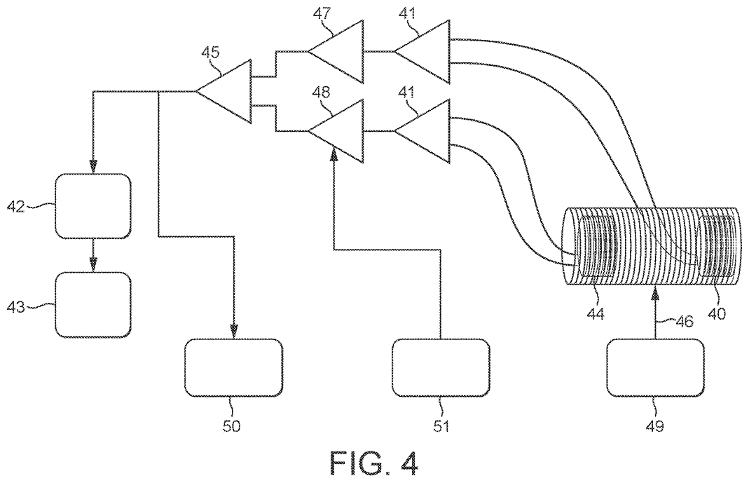

FIG. 4 shows a further preferred arrangement. This circuit operates on the same principle as the arrangement of FIG. 3, but uses a more sophisticated method of field cancellation, and passive coil matching. In particular, a known global magnetic field 44 is introduced to both coils 40, 44 to try to remove background magnetic field interference.

In this circuit, the outputs from the detection circuits 41 are passed through respective amplifiers 47, 48, respectively, before being provided to the differential amplifier 45. At least one of the amplifiers 47, 48 is tuneable. In use, a known global field 46, such as 50 or 60 Hz line (and harmonics) noise, or a signal, such as a 1 kHz signal, applied by a signal generator 49, is introduced to both coils 40, 44. The presence of a signal on this frequency on the output of the differential amplifier 45, which can be observed, for example, using an oscilloscope 50, will then indicate that the coils 40, 44 are not matched. An amplifier control 51 can then be used to tune the tuneable voltage controlled amplifier 48 to eliminate the global noise on the output of the differential amplifier 45 thereby matching the outputs from the two coils appropriately.

Most preferably in this arrangement, a known global field of 1 kHz or so is applied to both coils, so as to achieve the appropriate coil matching for the gradient subtraction.

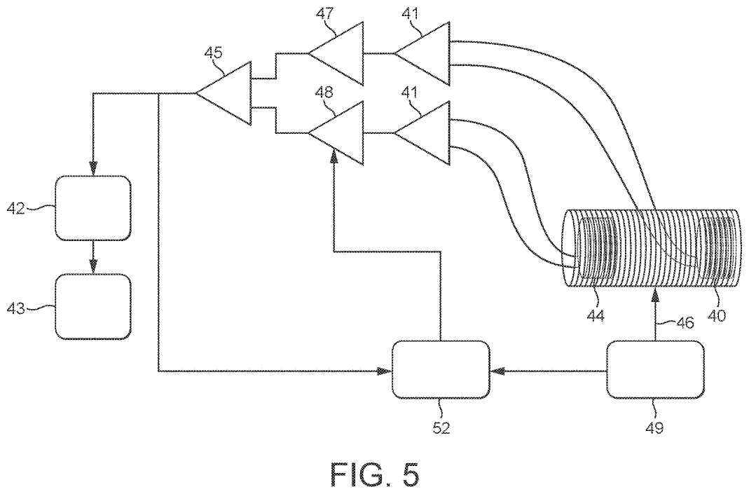

FIG. 5 shows a further variation on the FIG. 4 arrangement, but in this case using active coil matching. Thus, in this arrangement, the outputs of the coils 40, 44 are again channelled to appropriate detection circuits 41, and then to respective amplifiers 47, 48, at least one of which is tuneable. However, the tuneable amplifier 48 is tuned in this arrangement to remove the common mode noise using a lock in amplifier 52 or similar voltage controller that is appropriately coupled to the output from the differential amplifier 45 and the signal generator 49.

The above embodiments of the present invention show arrangements in which there is a single pickup coil that may be used to detect the magnetic field of the subject's heart. In these arrangements, in order then to make a diagnostic scan of the magnetic fields generated by a subject's heart, the single pickup coil can be moved appropriately over the subject's chest to take readings at appropriate spatial positions over the subject's chest. The readings can then be collected and used to compile appropriate magnetic field scans of the subject's heart.

It would also be possible to arrange a plurality of coil and detection circuit arrangements, e.g. of the form shown in FIG. 1, in an array, and to then use such an array to take measurements of the magnetic field generated by a subject's heart. In this case, the array of coils could be used to take readings from plural positions over a subject's chest simultaneously, thereby, e.g., avoiding or reducing the need to take readings using the same coil at different positions over the subject's chest.

FIGS. 6A and 6B show suitable coil array arrangements that have an array 60 of 16 detection coils 61, which may be then placed over a subject's chest to measure the magnetic field of a subject's heart at 16 sampling positions over the subject's chest. FIG. 6A shows a regular rectangular array and FIG. 6B shows a regular hexagonal array. In these cases, each coil 61 of the array 60 should be configured as described above and connected to its own respective detection circuit (i.e. each individual coil 61 will be arranged and have a detection circuit connected to it as shown in FIG. 1). The output signals from the respective coils 61 can then be combined and used appropriately to generate a magnetic scan of the subject's heart.

Other array arrangements could be used, if desired, such as circular arrays, irregular arrays, etc.

More (or less) coils could be provided in the array, e.g. up to 50 coils, or more than 50 coils. For example, where it is desired to measure the magnetic field of a different region of a subject's body (i.e. other than the heart), then an increased number of coils may be provided so as to provide an appropriate number of sampling points and an appropriate spatial coverage for the region of the subject's body in question.

It would also be possible in this arrangement to use some of the coils 61 to detect the background magnetic field for the purposes of background noise subtraction, rather than for detecting the wanted field of the subject's heart. For example, the outer coils 62 of the array could be used as background field detectors, with the signals detected by those coils then being subtracted appropriately from the signals detected by the remaining coils of the array. Other arrangements for background noise subtraction would, of course, be possible.

It would also be possible to have multiple layers of arrays of the form shown in FIG. 6, if desired. In this case, there could, for example, be two such arrays, one on top of each other, with the array that is closer to the subject's chest being used to detect the magnetic field generated by the subject's heart, and the array that is further away being used for the purposes of background noise detection.

To measure the magnetic fields generated by the heart, the above arrangements can be used to compile magnetic field scans of a subject's heart by collecting magnetic field measurements at intervals over the subject's chest. False colour images, for example, can then be compiled for any section of the heartbeat, and the scans then used, for example by comparison with known reference images, to diagnose various cardiac conditions. Moreover this can be done for significantly lower costs both in terms of installation and on-going running costs, than existing cardiac magnetometry devices.

FIG. 7 shows a typical ECG trace and the conventional labelling of the typical elements present in the ECG trace. Similar elements also occur in the MCG trace and the correspondence between the two has led to researchers using the same labelling convention.

As shown in FIG. 7, the ECG trace comprises a repeating P-P interval comprising a so-called P-wave, followed by a P-R (or P-Q) segment (where the combination of the P-wave and the P-R (or P-Q) segment is referred to as the P-R (or P-Q) interval), followed by a QRS complex, followed by an S-T segment, followed by a T-wave (where the combination of the S-T segment and the T-wave is referred to as the S-T interval, and the combination of the QRS complex and the S-T interval is referred to as the Q-T interval), followed by a T-P segment. Each of the features within the ECG can have diagnostic importance.

FIG. 8 shows an exemplary arrangement of the magnetometer as it is envisaged it may be used in a hospital, for example. The magnetometer 30 is a portable device that may be wheeled to a patient's bedside 31 where it is then used to take a scan of the patient's heart (e.g.). There is no need for any magnetic shielding, cryogenic cooling, etc. The magnetometer 30 can be used in the normal (non-shielded) ward environment. (Magnetic shielding and/or cooling could, however, be provided if desired.)

As used herein, a magnetometer or other apparatus in a “magnetically shielded” environment may comprise a magnetometer or other apparatus that is either arranged in a specially designed room or enclosure. In such arrangements, both the subject being measured and the equipment doing the measuring are contained within the same shielded enclosure. By contrast, as used herein, a magnetometer or other apparatus in a “non-magnetically shielded” comprises a magnetometer or other apparatus for which no external piece or pieces of apparatus are used to protect the subject being measured, nor the equipment doing the measuring.

The magnetometer system can be used in an analogous manner to detect and analyse other medically useful magnetic fields produced by other regions of the body, such as the bladder, abdomen, chest, head, brain, one or more foetuses, a muscle, etc.

Despite the success of MCG as a diagnostic aid, the hospital environment (such as an emergency department) can present challenges which interfere with the acquisition of acceptable MCG data.

Three main types of noise can cause such interference: homogenous noise (e.g. the earth's magnetic field), stochastic noise (e.g. white noise, coloured noise), and background noise (e.g. power line disturbances with power spectrum peaks at 50 or 60 Hz (and harmonics), vibrations of the system itself, etc.). Background noise can often exceed the MCG signal by orders of magnitudes and can vary in time, which makes its removal a challenging problem. The present embodiment is directed, in particular, to the removal of background noise components.

Background noise components may be characterised as being either low, medium or high frequency noise. Low frequency noise (0.1 to 1 Hz) is typically due to moving elevators, metal doors, metal chairs or other metallic objects. High frequency noise (>20 Hz) is mostly due to power supplies, monitor frequencies, or other electronic devices. Vibrations of the system itself cause disturbances in the middle frequency noise range (1 Hz to 20 Hz).

The Applicants have recognised that, where it is desired to take a scan of a patient's heart while the patient is on a hospital bed 31, noise can be produced due to coupling of residual magnetism in the steel frame of the hospital bed 31, which can vibrate due to the motion of the patient as their heart beats.

These transient noise pulses consist of a relatively short sharp initial pulse followed by decaying low-frequency oscillations. The initial pulse is due to the heart beat (systole), whereas the oscillations are due to the resonance of the system (bed and patient) excited by the initial pulse.

Once picked up by the magnetometer device 30, this transient noise can make it difficult to assess the quality of the captured data during the scanning process. It also makes it difficult to extract the useful undistorted magnetocardiograph data that is required for an accurate diagnostic.

“Ballistocardiographic noise” may be caused by vibration of the bed 31, where the vibration is correlated with the recoil forces of the body in reaction to cardiac ejection of blood into the vasculature.

Other sources of synchronised biological noise include, for example, “seismocardiographic noise” which may be caused by local vibrations of the chest wall in response to the heartbeat, as well as breathing, and changes in the position of the subject's body on the bed 31, e.g. due to talking, fidgeting, etc., that can cause vibration of the bed 31 which in turn can produce synchronous magnetic noise in the magnetocardiograph.

These biological sources of synchronous noise should be contrasted with other sources of noise such as nearby vibrations (e.g. vibrating lift shafts, large objects being dropped or moved, etc.). Although these other noise sources can cause vibration of the bed 31 which in turn can produce magnetic noise in the magnetocardiograph, generally such noise is not synchronised with the motion of region of the subject's body in question (e.g. heartbeat) and can therefore be reduced using averaging (over a long enough period of time).

Other support structures such as beds, chairs, etc., may also give rise to biological synchronous magnetic noise, e.g. where the support structure comprises a material that can produce a magnetic field, such as high permeability materials, and/or high conductivity materials.

High permeability materials include, for example, iron, steel, nickel, and various alloys thereof. High permeability materials comprise materials that can be magnetised and/or that can attract a magnet, e.g. ferrous materials that can generally hold and maintain a permanent magnetic field of their own (i.e. that are ferromagnetic). High permeability materials react strongly to applied magnetic fields, and are typically electrically conductive.

Low permeability, high conductivity materials comprise highly conductive materials that do not have a magnetic field of their own but can produce a response in reaction to changes in applied fields (e.g. paramagnetic or diamagnetic). Low permeability, high conductivity materials comprise, for example, stainless steel, aluminium, graphene, etc.

With respect to electrically conductive materials, if a conductive material is stationary and an applied field is stationary, it will not produce a magnetic field. However, if a conductive material is stationary and an applied magnetic field is moving, electric (eddy) currents may be induced in the material, which have their own corresponding magnetic fields. Equally, if a conductive material is moving and the magnetic field is stationary, electric (eddy) currents may be induced in the material, which have their own corresponding fields.

By contrast, low permeability, low conductivity materials may comprise, for example, wood, most plastics, ceramics, fiberglass, etc. Low permeability, low conductivity materials are (non-conductive) electrical insulators with low permeability and conductivity and do not provide any magnetic interference, i.e. do not produce magnetic fields even if they vibrate.

FIG. 9A shows cycle averaged MCG data for a healthy subject captured by a 37-channel magnetometer device in an unshielded environment on a wooden bed, and FIG. 9B shows corresponding data for a bed made of ferrous (magnetic) material. The averaged signal was filtered using a notch filter to remove power line noise followed by a finite impulse response (FIR) low pass filter.

The peaks visible in the middle of FIG. 9A correspond to the QRS section of the cardiac cycle (more specifically the time derivative of the magnetic field of the cardiac muscle and not the static field). These represent the highest signal to noise ratio part of the signal.

Distortions to the MCG data caused by the magnetic bed material are evident in FIG. 9B, making it impossible to extract useful information even from the QRS section.

FIG. 10A shows a log periodogram of raw MCG signals captured by a 37-channel magnetometer device in a non-shielded environment for a noise scan (i.e. without a subject present under the scan head). FIG. 10B shows a log periodogram of MCG signals for a healthy subject captured by a 37-channel magnetometer device in an unshielded environment for a subject on a wooden bed, and FIG. 100 shows a corresponding signal for a subject on a bed having ferrous (magnetic) material. A 8192-point Welch periodogram was used with a hamming window and a 4096-point overlap for the spectral calculations.

The noise peaks visible in FIG. 10A are due to the mains power supply and its subharmonics (50 Hz, 25 Hz, 16⅔ Hz etc.).

The contribution of the healthy subject MCG signal to the spectral content in FIG. 10B appears at approximately 4 Hz, 10 Hz and 33 Hz, while the contribution of the ballistic effects due to the bed material are clearly visible in the spectral content of FIG. 100.

These “ballistic effects” fall in the frequency range <10 Hz, making it difficult to extract useful information from the MCG signal.

The Applicants attempted to use a number of techniques to try to diminish or remove such unwanted noise from the MCG signal.

One such technique is nonlinear denoising (NLD) in state space. Nonlinear denoising operates on the reconstructed state space of the time series which represents the dynamical properties of the observed system. Background noise such as powerline disturbances fills a subspace in the state space which can be separated from the MCG manifold. This is done by recording the disturbances using an additional sensor, followed by a projection onto the noise subspace, followed by a subtraction from the original signal.

This approach requires the noise to be “at least approximately” deterministic, and therefore works well in removing powerline disturbances. However, the Applicants have found that this approach fails to remove transient noise caused by the bed vibrations, i.e. due to their nondeterministic nature.

In contrast with this, and in accordance with the present invention, the Applicants have found that a particular band-pass filter arrangement (as described further below) can be used to successfully separate the “ballistic effects” caused by the use of magnetic beds from the QRS complex. This allows a useful signal to be extracted from the corrupted MCG signal.

The Applicants have found, in particular, that a bandpass filter having a passband around 8-45 Hz can be used to separate the MCG signal from the ballistocardiographic (BCG) noise and background noise. The filter is designed to significantly reduce the impact of the ballistic effects from the measured signal, specifically the QRS region. The filter is a band pass filter constructed as combination of a high pass filter (removing ballistic effects <10 Hz), and a low pass filter (removing background noise >50 Hz).

FIG. 11 illustrates an ideal band-pass filter. An idealised filter is one that removes all frequency components above a given cutoff frequency, without affecting lower frequencies, and has linear phase response. All frequencies within the passband 10-50 Hz, are passed with unity amplitude, while all other frequencies are blocked. The passband is perfectly flat, the attenuation in the stopband is infinite, and the transition between the two is infinitely small. The filter's impulse response is a sinc function in the time domain, and its frequency response is a rectangular function. It is an “ideal” low-pass filter in the frequency sense, perfectly passing low frequencies, perfectly cutting high frequencies, and thus may be considered to be a “brick-wall” filter.

In the present embodiment, in order to approximate such an ideal filter, two windowed-sinc filters are combined to construct a band-pass filter that can separate the MCG signal from the BCG signal and background noise. This allows for an efficient separation of the QRS-complex from the ballistic effects and other background noise interferences, without phase distortions.

The filter is configured such that it removes all frequency components below a cut-off frequency f_{c1} and above a cut-off frequency f_{c2} without affecting frequencies in between. The filter is designed as the difference of two windowed-sinc filters whose cut-off frequencies are f_{c1} and f_{c2}. The filter is able to significantly reduce the impact of the ballistocardiographic effects (BCG), on the MCG signal, specifically the depolarisation (QRS) section.

In the present embodiment, the signal from the detector is firstly digitised, e.g. using a 4-bit 37-channel 2400 kS/s ADC. MCG signals are baseline corrected and averaged to increase signal-to-noise level. Data are averaged centring on the R wave peak, which is obtained using an accompanying ECG signal. The averaged signal can be windowed, using a suitable window function, to reduce abruptness.

FIG. 12A shows a filter kernel for a windowed-sinc filter, and FIG. 12B shows the frequency response (with a cut-off frequency of 45 Hz and M=2400). The filter acts as a low pass filter.

In the time domain, the filter kernel is a modification of the sinc function. The frequency response of the windowed-sinc filter is rectangular. This corresponds to the fact that the sinc filter is the ideal (brick-wall, i.e. rectangular frequency response) low-pass filter.

The windowed-sinc filter kernel with cut-off frequency f_{c1} is given by:

$${h}_{{f}_{c} [ i ]} = K \frac{sin ( 2 π {f}_{c} ( i - M / 2 ) )}{i - M / 2} w [ i ]$$

where w[t] is a window function centred on t=0, and where i ranges from 0 to M. The constant K is a normalisation factor chosen to provide a unity gain at zero frequency. The cut-off frequency f_{c} is expressed as a fraction of the sampling rate (a value between 0 and 0.5). The length of the filter kernel is determined by M, which must be an even integer.

The Applicants have found that, for the purposes of the present embodiment, the choice of window function is important. This involves a trade-off between roll-off and stop-band attenuation. Possible choices for the window function include the Hamming window, the Blackman window, the Bartlett window, and the Hanning window.

The Applicants have found in particular that, for the purposes of the present embodiment, the Blackman window is particularly suitable. This window has a slower roll-off compared with other window functions such as the Hamming window. However, the Blackman window has an improved stopband attenuation, and a lower passband ripple.

A Fourier transform may be performed to convert a signal in the time domain to its frequency domain counterpart. To calculate the output of the filter in the time domain a convolution may be performed, and in the frequency domain a point-by-point multiplication may be performed.

The length of the filter kernel M determines the transition bandwidth of the filter in the frequency domain, BW (the transition bandwidth is measured from where the curve leaves a value of one, to where it almost reaches zero), and is expressed as a fraction of the sampling rate (i.e. a value between 0 and 0.5). The trade-off between the computation time (which depends on the value of M) and the filter sharpness (the value of BW) can be expressed through the approximation:

$$M \approx \frac{4}{BW}$$

As such, the sharper the filter is (the smaller the transition bandwidth BW), the longer is the time required to perform convolution in the time domain.

In the present embodiment, the signal is averaged into a single 1s cycle (e.g. 2400 samples at 2400 Hz sampling rate, i.e. one second of data which is approximately equivalent to a single heartbeat). The length of M is then maximised M=2400 to minimise the transition bandwidth BW. This means that BW˜ 4/2400˜0.00167, or BW˜2 samples.

The filter of the present embodiment is constructed as the difference of two windowed-sinc filters whose cut-off frequencies are f_{c1} and f_{c2}. Since taking linear combinations in the frequency domain is equivalent to taking the same linear combinations in the time domain, the filter is constructed as the difference of two windowed-sinc filters:

g[i]=h_{f}_{_{c2}}[i]−h_{f}_{_{c1}}[i]

This provides a band pass filter which only passes frequencies between f_{c1} and f_{c2}. If f_{c2} is set as 0.5, a high pass filter is obtained, and if f_{c1} is set as 0.0, a low pass filter is obtained.

FIG. 13A shows the filter kernel and FIG. 13B shows the frequency response of the difference of two windowed-sinc filters with cut-off frequencies f_{c1}=0.0033 (8.0 Hz), f_{c2}=0.01875 (45.0 Hz) and M=2400. The filter acts as a band pass filter.

The filter can be applied in either the time domain or the frequency domain to effectively separate the repolarisation (QRS section) of the MCG signal from the BCG effects and background noise.

FIG. 14 illustrates example MCG data for a healthy subject on a metal bed obtained in a non-shielded room.

FIG. 14A shows the obtained averaged MCG signal, and FIG. 14B shows the frequency spectrum (Fourier spectrum) of the signal. FIGS. 14C and 15D respectively show the filter kernel and frequency response of the difference of two windowed-sinc filters with cut-off frequencies of f_{c1}=0.0033 (8.0 Hz) and f_{c2}=0.01875 (45.0 Hz) with M=2400.

FIGS. 14E and 14F show the time series and its corresponding Fourier spectrum resulting from the present filtering method (solid line). FIG. 14E also shows the result of applying a filter with cut-off frequencies at 2 Hz and 45 Hz to the signal of FIG. 14A (dashed line), where the presence of ballistic noise is evident.

FIG. 15 illustrates the effectiveness of the windowed sinc filter in removing the ballistocardiographic effects due to the ferrous (magnetic) material of the bed. FIG. 15A shows the data of FIG. 9B, and FIGS. 15B and 15C show the data after the filter has been applied. A windowed-sinc filter kernel with a Blackman window and cut-offs at 8 Hz and 45 Hz was used. The ballistocardiographic effects have been removed from the signal and the useful MCG features (namely the QRS section) is now visible and can be used to extract medically useful information.

FIG. 16 shows a sequence of data processing steps in accordance with the present embodiment.

A sensor 40 and a digitiser 42 are used to obtain a signal 101. The signal is then averaged 102 over plural periods. This involves using a trigger such as the ECG to determine the plural repeating periods of the signal. Data is taken from the target waveform in each of plural windows around each of plural triggers. Several subsequent windows are averaged to remove random noise.

Filtering 103 is then applied to remove the noise that cannot be removed by averaging, i.e. the bed noise and other background noise as described above.

Following any additional data processing 104, diagnostic parameter extraction 105 may be performed, and used for analysis 106.

Some examples of medically useful signals that may be analysed are (i) S-T baseline shifts (STEMI) e.g. S-T elevated myocardial infarction; and (ii) R-S transition rate, e.g. bundle branch block. However, in general any of the signal features described herein may have diagnostic importance and may be used for analysis.

It can be seen from above that the present invention provides an improved magnetometer system for medical use. This is achieved, in the preferred embodiments of the present invention at least by filtering a signal or signals from using a filter that is configured to attenuate synchronised, e.g. ballistocardiographic noise.

1. A method of using a magnetometer system to analyse the magnetic field of a region of a subject's body, the method comprising:
using one or more detectors to detect the time varying magnetic field of a region of a subject's body;
filtering a signal or signals from the one or more detectors using a filter or filters, wherein the filter or filters is configured to attenuate noise in the signal or signals that is synchronised with motion of the region of the subject's body; and
using the filtered signal or signals to analyse the magnetic field generated by the region of a subject's body.

2. The method of claim 1, wherein the filter or filters is configured to attenuate signals having frequencies below a low frequency cut-off frequency, wherein the low frequency cut-off frequency is optionally between around 8 and 12 Hz.

3. (canceled)

4. (canceled)

5. The method of claim 1, wherein the filter or filters is configured to attenuate signals having frequencies above a high frequency cut-off frequency.

6. The method of claim 5, wherein the high frequency cut-off frequency is between around 45 and 60 Hz.

7. The method of claim 1, wherein the filter or filters comprises at least one windowed sinc filter.

8. The method of claim 7, wherein the windowed sine filter is formed using a Blackman window.

9. The method of claim 1, comprising using the magnetometer system to detect the time varying magnetic field of a region of a subject's body in a non-magnetically shielded environment.

10. The method of claim 1, comprising using the magnetometer system to detect the time varying magnetic field of a region of a subject's body when the subject is supported by a structure that comprises electrically conductive and/or ferrous material.

11. The method of claim 1, wherein the region of the subject's body comprises one of: the abdomen, bladder, heart, head, brain, chest, womb, one or more foetuses, or a muscle.

12. A magnetometer system for medical use, comprising:
one or more detectors for detecting the time varying magnetic field of a region of a subject's body; and
a filter or filters configured to filter a signal or signals from the one or more detectors, wherein the filter or filters is configured to attenuate noise in the signal or signals that is synchronised with motion of the region of the subject's body;
wherein the magnetometer system is configured to provide the filtered signal or signals for use to analyse the magnetic field generated by the region of the subject's body.

13. The system of claim 12, wherein the filter or filters is configured to attenuate signals having frequencies below a low frequency cut-off frequency.

14. The system of claim 13, wherein the low frequency cut-off frequency is between around 8 and 12 Hz.

15. A magnetometer system for medical use, comprising:
one or more detectors for detecting the time varying magnetic field of a region of a subject's body; and
a filter or filters configured to filter a signal or signals from the one or more detectors, wherein the filter or filters is configured to attenuate signals having frequencies below a low frequency cut-off frequency, wherein the low frequency cut-off frequency is between around 8 and 12 Hz;
wherein the magnetometer system is configured to provide the filtered signal or signals for use to analyse the magnetic field generated by the region of the subject's body.

16. The system of claim 12, wherein the filter or filters is configured to attenuate signals having frequencies above a high frequency cut-off frequency.

17. The system of claim 15, wherein the high frequency cut-off frequency is between around 45 and 60 Hz.

18. The system of claim 12, wherein the filter or filters comprises at least one windowed sinc filter.

19. The system of claim 12, wherein the windowed sinc filter is formed using a Blackman window.

20. The system of claim 12, wherein the region of the subject's body comprises one of: the abdomen, bladder, heart, head, brain, chest, womb, one or more foetus, or a muscle.

21. The system of claim 12, wherein the magnetometer system is configured to detect the time varying magnetic field of a region of a subject's body in a non-magnetically shielded environment.

22. The system of claim 12, further comprising a support structure for supporting the subject's body, wherein the support structure comprises electrically conductive and/or ferrous material.
